(19) United States
(12) Patent Application Publication
(10) Pub. No.: US 2022/0354564 A1
(43) Pub. Date: Nov. 10, 2022

(54) ARTICULATED ENDOSCOPIC INSTRUMENT

(71) Applicant: The Cleveland Clinic Foundation, Cleveland, OH (US)

(72) Inventors: Tolga Erim, Fort Lauderdale, FL (US); Tanju Erim, Coconut Creek, FL (US); Shengqiang Gao, Beachwood, OH (US)

(21) Appl. No.: 17/738,257

(22) Filed: May 6, 2022

Related U.S. Application Data
(60) Provisional application No. 63/184,851, filed on May 6, 2021.

(51) Int. Cl. A61B 18/08; A61B 18/10

(52) U.S. Cl. CPC A61B 18/082; A61B 18/10; A61B 2018/00101

(57) ABSTRACT

An endoscopic instrument containing a retractable tool at its end that is articulated by engaging an actuator that moves pull wires to angularly move the tip of the instrument along a plane for conducting a procedure at a target site. The tool of instrument is linearly move from an extension position to a retracted position by another actuator. The instrument provide rotational control of the tool and instrument tip portion with a rotational actuator for rotating housings that carry both pull wires and a conducting wire connected to the tool.

This application claims priority to and any other benefit of U.S. Provisional Patent Application Ser. No. 63/184,851 filed May 6, 2021, the contents of which are incorporated herein in their entirety by reference.

### TECHNICAL FIELD

The present invention relates to an articulated endoscopic instrument and, in particular, an endoscopic dissection knife capable of precise articulation along a plane at or near its distal end.

### BACKGROUND

During surgical procedures, such as endoscopy and laparoscopy, in which a targeted site for the application of a tool or instrument is difficult to access, the instruments must be steered within or towards a target organ or tissue from a position outside the body. Examples of endoscopic procedures include sigmoidoscopy, colonoscopy, esophagogastroduodenoscopy, and bronchoscopy. Traditionally, an end portion of an endoscopic instrument is advanced by pushing it forward and retracted by pulling it back. The instrument can also be manually moved side to side to achieve lateral movement of the device. The tip of the device, such as a knife or blade, may be directed by twisting and general up/down and left/right movements. Oftentimes, this limited range of motion makes it difficult to negotiate acute angles in small areas which can create patient discomfort and increase the risk of trauma to surrounding tissues.

The present invention provides an instrument capable of precise articulation near its distal end to assist the user in performing procedures. The articulation reduces or eliminates the need to pull or push the instrument back and forth or perform up/down and left/right movements to position the tip of the instrument in the desired position.

### SUMMARY

In a first aspect, disclosed is an endoscopic instrument that includes a conductive center wire having a first end and a second end and a tool secured to the second end of the center wire, a first actuator secured to the first end of the center wire and configured to move the center wire in a linear manner back and forth, one or more pull wires each having a first end and a second end, and a second actuator secured to the first end of the one or more pull wires and the second end of the one or more pull wires secured to an end section of the instrument housing the tool; and the second actuator is configured to angularly articulate the tool in a single plane by moving the one or more pull wires to change the position of the end section.

In an example of aspect 1, the second actuator can move the end section and tool up to 90 degrees in a single plane.

In another example of aspect 1, the second actuator is secured to a second pull wire having a first end and a second end. The second actuator is secured to the first end of the second pull wire and the end section of the instrument is secured to the first end of the second pull wire.

In another example of aspect 1, the second actuator is configured to angularly articulate the end section and tool ±90 degrees in a single plane by moving the one or more pull wires. The arc radius of the angular articulation of the end section and the tool in the single plane can be in the range of 0.2 to 10 centimeters, 0.4 to 5 centimeters or 1 to 3 centimeters.

In another example of aspect 1, the one or more pull wires are in direct contact with guides for positioning the pull wires at 180 degrees relative to one another to form a plane in which an end section of the instrument can angulate. The guides are positioned on an end of a guide section connected to an elongated housing containing the one or more pull wires.

In another example of aspect 1, the center wire is an electrode.

In another example of aspect 1, the tool is an electrocautery knife, a snare or a clip.

In another example of aspect 1, a portion of the center wire and a portion of the one or more pull wires are positioned in an elongated housing, for example a coiled housing, and the elongated housing is covered by an insulating sheath.

In another example of aspect 1, the instrument further includes a rotation actuator configured to rotate an end section housing the tool, the end section has an open end configured to pass the tool therethrough and the open end defining the end face of the instrument.

In another example of aspect 1, the center wire includes a flexible section positioned near a second end of the one or more pull wire. The flexile section of the center wire is further secured to an end portion of the center wire, which is secured to the tool.

In another example of aspect 1, the instrument further includes an electrical coupler for coupling the center wire to an electrical source. In an example, the electrical coupler is positioned adjacent or near the first actuator.

In another example of aspect 1, a spring blade is positioned between a guide section secured to the end of the elongated housing and the end section, and the spring blade is articulated ±90 degrees in a single plane by moving the one or more pull wires.

In a second aspect, there is an endoscopic instrument that includes the following features: an elongated housing having a first end and a second end, a center wire having a first end and a second end and a portion of the center wire positioned in the elongated housing, a first actuator secured to the first end of the center wire and the first actuator configured to move the center wire in a linear manner, a tool secured to the second end of the center wire and the tool configured to pass in and out of a first end of an end section by use of the first actuator, a pair of pull wires, each pull wire having a first end and a second end and a portion of the pair of pull wires positioned in the elongated housing, a second actuator secured to the first end of each of the pair of pull wires and the second end of each of the pair of pull wires secured to a second end of the end section, and the second actuator is configured to angularly articulate the tool ±90 degrees in a single plane by moving the pair of pull wires.

In an example of aspect 2, the elongated housing is a coiled metal housing.

In another example of aspect 2, the elongated housing is secured to a handpiece containing the first and second actuators at one end and the opposite end of the housing is secured to a guide section having two guides for the pair of pull wires.

In another example of aspect 2, the pair of pull wires are in direct contact with guides for positioning the pair of pull wires at 180 degrees relative to one another and to form a plane in which the end section can angulate.

In another example of aspect 2, the guides are secured to a guide section, the guide section connected to the second end of the elongated housing such that the pair of pull wires pass through the elongated housing and into the guide section.

In another example of aspect 2, a spring blade is positioned between a guide section and the end section of the instrument, the spring blade can be articulated ±90 degrees in a single plane by moving the pair of pull wires.

In another example of aspect 2, the spring blade has a first end and a second end, the first end of the spring blade secured to the second end of the end section and the second end of the spring blade secured to the first end of a guide section.

In another example of aspect 2, the spring blade has a first end and a second end, the first end of the spring blade extends into the second end of the end section and the second end of the spring blade extends into the first end of a guide section.

In another example of aspect 2, the spring blade is elastic and configured to return to its unarticulated shape.

Any one of the above aspects (or examples of those aspects) may be provided alone or in combination with any one or more of the examples of that aspect discussed above; e.g., the first aspect may be provided alone or in combination with any one or more of the examples of the first aspect discussed above; and the second aspect may be provided alone or in combination with any one or more of the examples of the second aspect or first aspect discussed above; and so-forth.

Additional features and advantages will be set forth in the detailed description which follows, and in part will be readily apparent to those skilled in the art from that description or recognized by practicing the embodiments as described herein, including the detailed description which follows, the claims.

### BRIEF DESCRIPTION OF THE DRAWINGS

The above and other features, examples and advantages of aspects or examples of the present disclosure are better understood when the following detailed description is read with reference to the accompanying drawings, in which:

FIG. 1. illustrates an example endoscopic instrument in accordance with an embodiment.

FIG. 2. illustrates an example actuator portion of an endoscopic instrument in accordance with an embodiment.

FIG. 3. illustrates an example actuator portion of an endoscopic instrument in accordance with an embodiment.

FIG. 4. illustrates a retracted position of an endoscopic instrument in accordance with an embodiment.

FIG. 5. illustrates an extended position of an endoscopic instrument in accordance with an embodiment.

FIG. 6 illustrates a first angulated position of a tool of an endoscopic instrument in accordance with an embodiment.

FIG. 7 illustrates a second angulated position of a tool of an endoscopic instrument in accordance with an embodiment.

FIG. 8 illustrates a tip portion of an endoscopic instrument in accordance with an embodiment.

FIG. 9 illustrates a tip portion of an endoscopic instrument in accordance with an embodiment.

FIG. 10 illustrates a tip portion of an endoscopic instrument in accordance with an embodiment.

FIG. 11 illustrates a spring blade for use with a tip portion of an endoscopic instrument in accordance with an embodiment.

FIG. 12 illustrates a spring blade for use with a tip portion of an endoscopic instrument in accordance with an embodiment.

### DETAILED DESCRIPTION

Example embodiments are described and illustrated herein. These illustrated examples are not intended to be a limitation on the present embodiments. For example, one or more aspects of the system can be utilized in other embodiments and other types of instruments. Such systems may, however, be embodied in many different forms and should not be construed as limited to the embodiments set forth herein. Like, but not necessarily the same, elements in the various figures are denoted by like reference numerals for consistency. Terms such as “first,” “second,” “front,” and “rear” are used merely to distinguish one component (or part of a component or state of a component) from another. Such terms are not intended to denote a preference or a particular orientation.

Herein, when a range such as 5-25 (or 5 to 25) is given, this means preferably at least or more than 5 and, separately and independently, preferably not more or less than 25. In an example, such a range defines independently 5 or more, and separately and independently, 25 or less.

The present disclosure relates to an endoscopic instrument that provides articulation of a tool such as an electrocautery knife at its tip or distal end. Electrocautery uses electrical current to heat a conductive wire that is then applied to target tissue in order to burn or coagulate a specific area of tissue. Heat is passed through a resistant metal wire which is used as an electrode. This hot electrode is then placed directly onto the treatment area destroying that specific tissue. Such endoscopic submucosal surgeries benefit from a high degree of instrument mobility very close to the target site. Presently, ablation instruments have ablation tools with very limited mobility and require a large area to operate. The endoscopic instrument disclosed herein allows controlled movement near the end of the instrument, similar to bending the furthest joint on one finger as opposed to bending the whole finger. By providing a defined bend moment near the end of the instrument, the instrument can be operated with reduced movement around the target site and lessen radially impact at the target site in a patient during procedures. In addition, a bending moment near the end of the instrument provides for more precise control at the target site leading to a more efficient and successful result. The instrument also can be sized such that it can pass through working lumens of an endoscope along with other instruments, as necessary. For instance, the main body of the endoscopic instrument, for example, the insulated elongated housing and tip portion containing the tool, can have outer diameters in the range of 1 to 4 mm.

As described herein, the endoscopic instrument can be used in connection with an endoscopic submucosal dissection (ESD) or electrocautery (e-knife) knife and can angulate up to 180 degrees at the tip portion of the instrument in a single plane, optionally rotate up to 360 degrees, and extend and retract through an end section or standard channel gastroscope. The tool (e.g., knife) has a retractable tip connected to a conducting center wire, which is linearly moveable and connected to an electrical source for transmitting electrical current to the tool. To enhance flexibility and angular movement of the tip portion of the instrument housing the tool, from an articulated to the unarticulated position or the base position, a flexible spring blade can be optionally arranged in the instrument near the tool and end portion one or more pull wires. Angulation of the end section of the instrument and tool is controlled by moving and tensioning one or more pull wires that can be positioned along with the center wire along the length of the instrument, for example, within a channel centrally located within an elongated housing and outer plastic insulating skin surrounding or coating the housing. The operator moves the tip of the tool by rotating a directional actuator knob, which controls the movement of directional pulling wires and the instrument end. More specifically, the rotation movement of a knob or disc directs the movement of the pull wires to change the angular position of the tip portion of the instrument. As such, surgeries performed with the instrument provide surgeons with increased control over the dissection and an overall increase in safety and length of procedure.

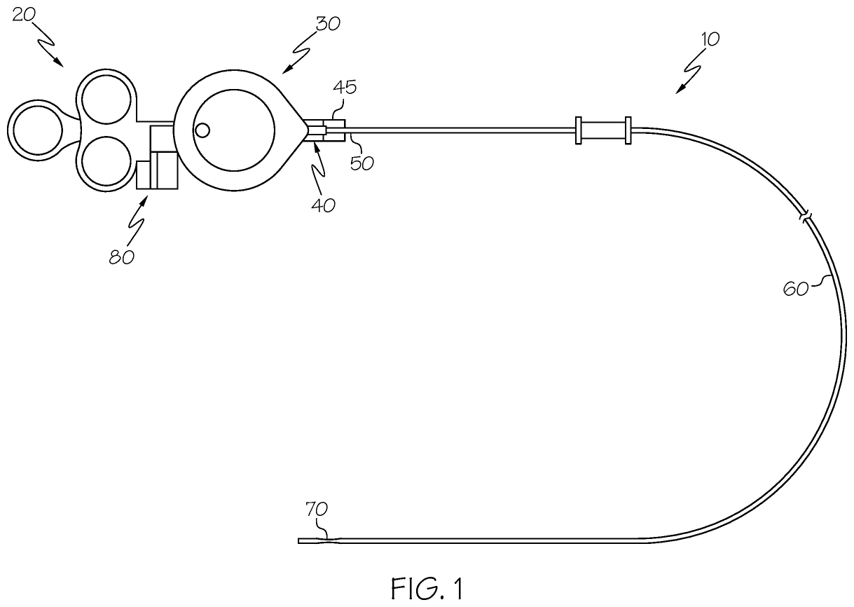

The figures illustrate various embodiments and features of the endoscopic instrument. FIG. 1 shows an example endoscopic instrument 10. As will be described and illustrated herein, the endoscopic instrument 10 comprises a retractable tool, e.g., an electrocautery knife, at its distal end. The endoscopic instrument 10 includes a handpiece, which is used by the surgeon during a procedure to hold the instrument and manually control various movements of the tool. Movement of the tool by using the handpiece can include linear (back and forth in an extended and retracted position), rotational, and angular in a single plane. The handpiece includes a first actuator 20. The first actuator 20 is configured to move a center wire, or electrode, in a linear manner to extend and retract from a distal end of the instrument. During procedures, the center wire and tool attached thereto is extended from the end of the instrument and then can be retracted into a housing or end section of the instrument for post-operative storage.

The first actuator 20 can include any suitable configuration, for example, one or more loops for accommodating a user's hand and fingers for gripping the instrument and moving the first actuator during operation. In an example, the first actuator can include one or more fixed finger loops positioned adjacent a slider. The slider, attached to one end of the center wire, can slide along a shaft or coupler for moving the center wire in a linear motion to extend and retract the tool from the end of the instrument. In another example, as shown in FIG. 1, the first actuator 20 can include two fixed finger loops for gripping the instrument and one moveable or sliding finger loop or handle that is attached to one end of the center wire for adjusting the center wire in a linear manner.

The handpiece further includes a second actuator 30. The second actuator 30 is configured to manipulate a position of a tool coupled to an end of the center wire of the instrument, such as an electrocautery knife, in an angular manner by movement of a tip portion 70 of the instrument. The second actuator 30 can include any suitable configuration, for example, a rotatable disc with a gripping feature or knob. The instrument is also fitted with a connector 40 that is used to couple the first actuator 20 and the second actuator 30 to a first end 50 of a flexible elongated housing 60, for example a coiled metal tube. The connector 40 is generally a hollow insulating member made of a plastic or other suitable material and can include one or more portions to provide a suitable connection between the handpiece and the elongated housing 60 that houses and guides the center wire and pull wires. The connector 40 can further include a rotation actuator 45 at one end. The rotation actuator 45 is secured to a first end 50 of the elongated housing 60, for example, near the second actuator 30 as shown in FIG. 1. The rotation actuator 45 can turn clockwise or counterclockwise to rotate the elongated housing 60, which is fixed to a tip portion 70 of the instrument. As elongated housing 60 rotates by turning the rotation actuator 45, the tool at the distal end of the instrument likewise rotates thus providing rotational control of the tip portion and tool to the user.

Regarding the tool for use with the instrument, it is to be appreciated that other surgical tools, such as a loop electrode, electrosurgical scalpel, or any other suitable tip can be implemented and connected to the center wire. It is to be further appreciated that the tool described herein can be used in an electrosurgery application, if desired. As further shown in FIG. 1, an electrical coupler 80 can be positioned at any suitable location in the handpiece area, for instance an electrical coupler 80 at or near the first actuator portion 20 for coupling the center wire to an electrical source (not shown) for providing an electrical current to the tool positioned at the other end of the center wire.

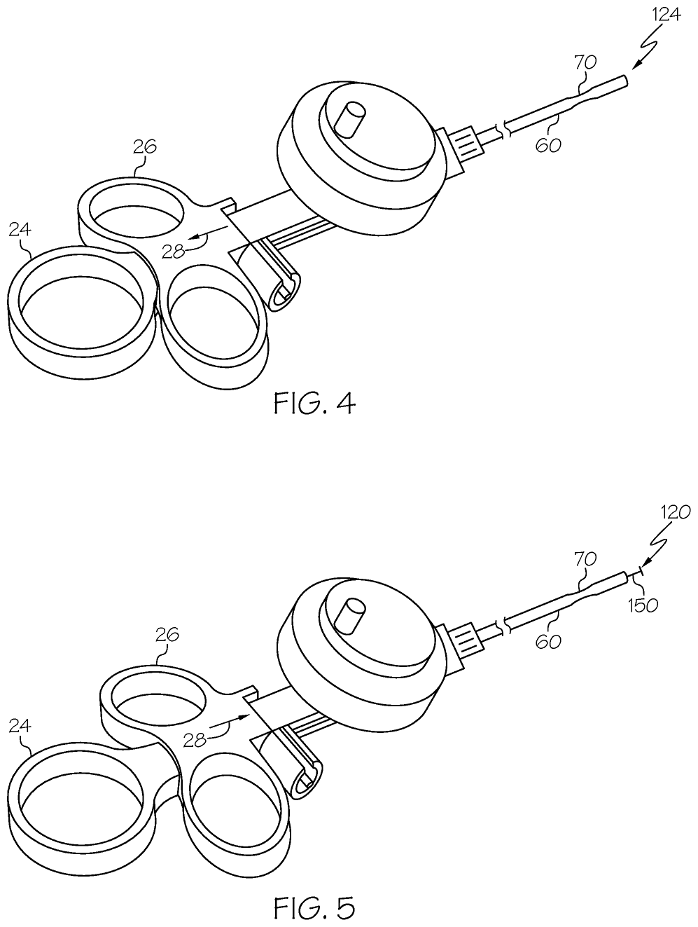

Turning to FIG. 2, the handpiece of the endoscopic instrument 10 is shown in greater detail. The first actuator 20 includes a fixed handle 24 and a moveable operation handle 26, both can include at least one finger loop as shown, which facilitates one-handed operation of the first actuator 20 by a user. The operation handle 26 is secured to a first end of the center wire that extends to the tip portion of the instrument through elongated housing 60. A second end of the center wire is secured to a first end of the tool. As shown in FIGS. 4 and 5, movement of the first actuator (e.g., operation handle 26) towards and away from the fixed handle 24 in the directions shown by arrow 28 selectively extends or projects, FIG. 5, 120, and retracts, FIG. 4, 124, the tool 150 from the tip portion 70 of the instrument. As the operation handle 26 is moved towards the fixed handle 24, the tool is projected from the tip portion 70 and ready for use and thus the first actuator 20 provides for a linear movement of the tool within the elongated housing 60. Likewise, the tool 150 is retracted with the operation handle 26 into the tip portion 70 housing to protect and store the tool. Movement of the first actuator 20 or operation handle 26 can be in the range of 0.5 to 10 centimeter (cm) or 1 to 5 cm depending on the desired extension length of the tool from the end of the instrument.

Also shown in FIG. 2, the second actuator 30 of the handpiece includes a housing 34, which houses first end portions of one or more or a pair of pull wires, a rotatable portion 36 that operates to selectively pull one of pull wires at a time depending if the rotatable portion 36 is turned clockwise or counterclockwise. That is, the rotatable portion 36 is secured to, directly or indirectly, the ends of the pull wires. For example, the ends of the pull wires can be secured to a disc that is controlled by rotatable portion 36 for tensioning and moving the pull wires that selectively articulate the end section and tool of the instrument. The rotatable portion 36 can take the form of a single knob or disc or any other suitable structure and includes a protrusion or gripper 38 that can be used for easy manipulation of the rotatable portion 36. The protrusion 38 may also provide a quick visual indication of a position of the distal end of the endoscopic instrument.

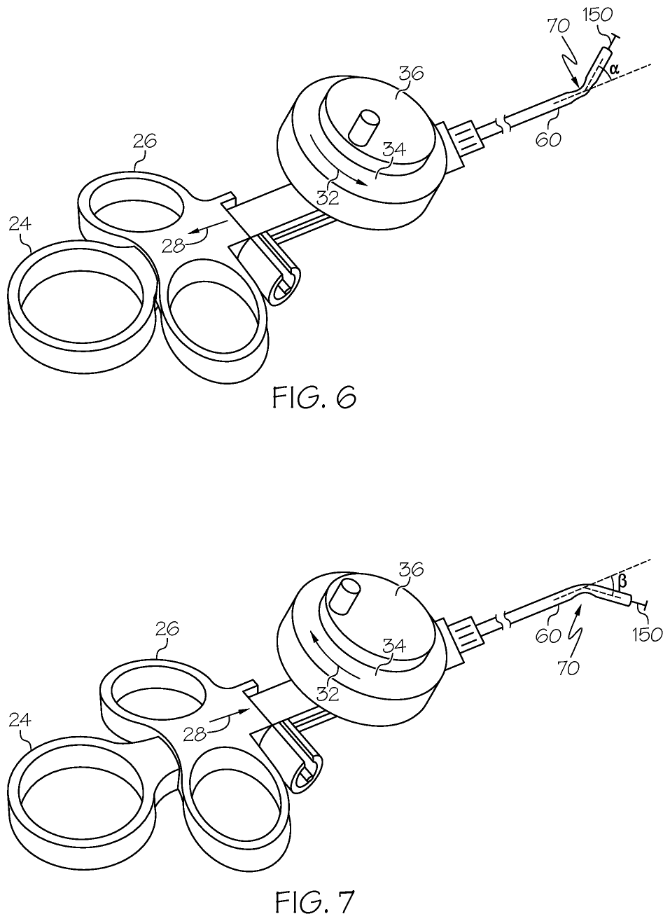

FIGS. 6 and 7 illustrate an example rotation of the rotatable portion 36 with respect to housing 34 for actively positioning the instrument tip portion 70 and in turn, the tool (e.g., electrocautery knife) extending from the tip portion 70 of the endoscopic instrument 10. When the rotatable portion 36 is turned in a first counterclockwise direction, as shown in FIG. 6, the end section of tip portion 70 is angled in a first direction from a single plane (shown as dotted line). The angle α of the tip portion 70 and tool 150 with respect to a longitudinal axis of the single plane of the elongated housing 60 is controlled by and dependent on the rotation amount in the counterclockwise direction of rotatable portion 36. In a similar manner, when the rotatable portion 36 is turned in a second clockwise direction, as shown in FIG. 7, the end section of tip portion 70 and tool 150 is angled in an opposition direction. The angle β of the tip portion with respect to the longitudinal axis of the single plane of the elongated housing 60 is controlled by and dependent on the rotation amount in the clockwise direction. Thus, movement in both the α- and β-angle directions, relative to the main portion of the elongated housing 60 can be achieved when the single rotatable portion 36 is both maneuvered and coupled to one or more pull wires. Movement of only one of the α- or β-angle directions can be achieved when the rotatable portion 36 is coupled to a single pull wire and thus tensioning of the pull wire causes one articulation direction. In the instance only one pull wire is used, the rotation actuator 45 can further position the end section of the tip portion 70, and, in turn, the tool 150 secured to the center wire, in a 360-degree rotation at the target site. Accordingly, movement of the second actuator portion 30, 31 and/or rotation actuator 45, 46 can control a tip section of the endoscopic instrument such that the tip section can rotate fully through about 180-degrees in a single plane with precision, e.g. plus or minus 90-degrees in either direction along a single plane.

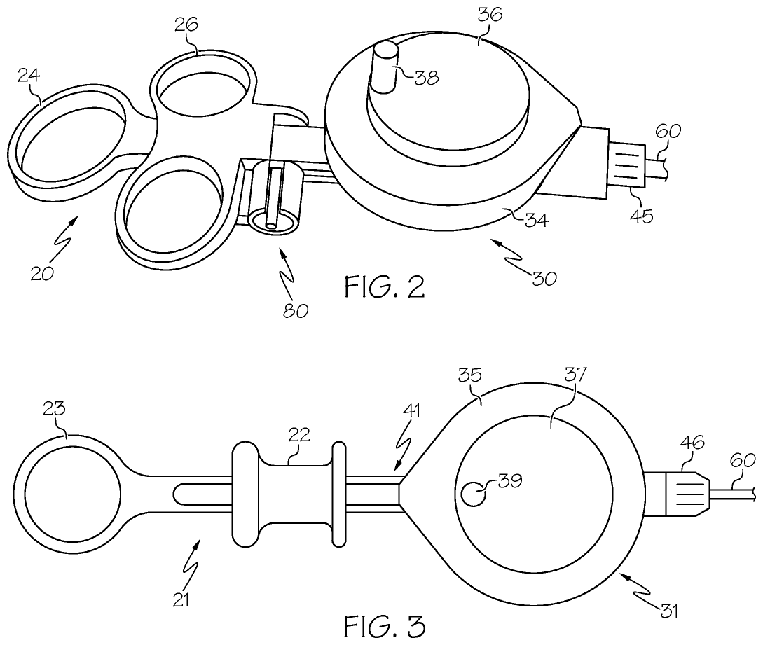

FIG. 3 shows another embodiment of an example endoscopic instrument 10 having a first actuator 21, a second actuator 31, a connector 41 and a rotation actuator 46. The first actuator 21 has a single, fixed finger loop 23 positioned adjacent a moveable slider 22 for linearly moving a center wire connected thereto and to a tool connected to an opposite end of the center wire at the end of the instrument. A user moves slider 22 up and down along connector 41, which can travel from finger loop 23 to the top of second actuator 31 housing, to either extend or retract the tool from the instrument tip portion during a procedure. The instrument can further include an electrical coupler, for example, positioned at any suitable location in the handpiece area or on connector 41, for coupling the center wire to an electrical source for supplying electrical current to the tool (not shown).

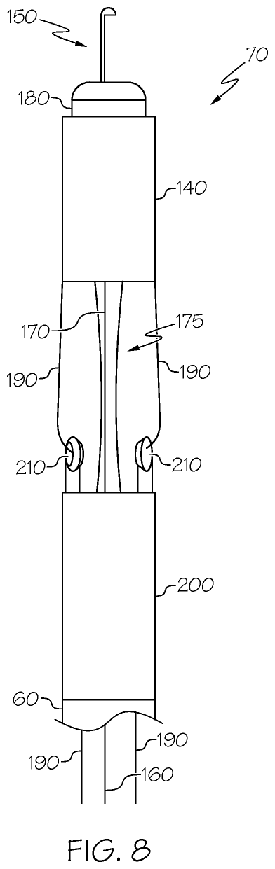

The second actuator 31 is similar to the second actuator 30 of FIG. 2. The second actuator 31 is configured to change the position of the tool coupled to the center wire of the instrument in an angular manner by movement of an end section of a tip portion of the instrument (e.g. end section 140 of FIG. 8). The second actuator 31 secures in a housing 35 one or more pull wires that can be secured to a rotating disc or similar structure engaged with a rotatable or moveable portion 37 to actuate a pull wires and thereby selectively control the angular position of the tool. The rotatable portion 37 can be turned clockwise or counterclockwise to tension or move a pull wire for actuating the end of the instrument and tool at an angle. The rotatable or moveable portion 37 can be in the form of a knob, disc or other suitable structure and can further include a protrusion or gripper 39 for a user to engage and manipulate portion 37. Protrusion 39 may also provide a visual indication to a user of a position of the distal end of the endoscopic instrument and tool during procedures.

As shown in FIG. 3, positioned adjacent the second actuator 31 is a rotation actuator 46 configured to rotate the elongated housing 60 to which it is fixed and, in turn, rotating an end portion of the instrument. As the elongated housing 60 rotates by twisting the rotation actuator 46, the tool at the distal end of the instrument rotates up to 360 degrees, either in a clockwise or counterclockwise direction. The rotation actuator 46 is secured to connector 41 and contains a moveable piece that can turn clockwise or counterclockwise depending on the direction of rotation desired for the tip portion of the instrument. To facilitate rotation of the elongated housing 60, rotation actuator 46 is secured to the housing such that turning actuator 46 directly turns or rotates the elongated housing and tip portion of the instrument. Securing the rotation actuator 46 to the elongated housing can be accomplished by any suitable means, for example, adhesive, a clamp, compression fitting, etc.

FIG. 8 illustrates a detailed view of the tip portion 70 of the endoscopic instrument 10 in accordance with the present embodiment. The tip portion 70 include an end section 140 having two ends, a passage therethrough, and an opening at each end. Extending from the distal or first end of the end section 140 is a tool 150, which can be extended and retracted by a first actuator moving a center wire 160 in a linear manner. The center wire 160 passes through elongated housing 60 and into end section 140, all of which can be covered with an insulating sheath to protect against any electrical current along the surface of the instrument. A ceramic insulator 180 is provided adjacent the opening at the first end of the end section 140 and the tool 150. The tool 150 can include any desired configuration depending upon the procedure desired. For instance, the tool 150 can be a knife that can be pointed, flat, widened and flat like a nail, offset, etc. In other examples, the tool 150 can be any suitable tool, for instance, a snare or a clip or grasper. One end of the tool 150 is secured, for example by a weld or the like, to the end of moveable center wire 160 that can, in certain instances, pass through end section 140 for providing electrical current to the tool.

The center wire 160 extends further upstream of tool 150 through guide section 200 and back to the actuator through the elongated housing. The center wire 160 can be any suitable conductive wire for providing electrical current to the tool. The center wire 160 can be a single monofilament or a wire made up of multiple filaments, for example, filaments twisted or combined together in any configuration. In another example, the center wire 160 can be made up of multiple sections of wire such that at least two of the sections are different from one another. For instance, the main portion of the center wire connected to the first actuator and extending to the tip portion and through the guide section 200 can be a monofilament, which is connected to a flexible portion of a braided or twisted wire of multiple filaments positioned between the guide section 200 and the end section 140, which is further connected to another monofilament section at or near end section 140 and connected at one end to tool 150. By incorporating a flexible section in the center wire between guide section 200 and end section 140, actuation of the end section is enhanced as the pull wires 190 are engaged with the second actuator to angulate end section 140 and tool 150.

As further shown in FIG. 8, a pair of pull wires 190, which are connected to a second actuator (not shown), extend through guide section 200 that is secured to the end of elongated housing 50. The guide section 200 can include one or more guides 210 for positioning one or more pull wires 190 away from one another at a predetermined distance. The guides 210 can be any suitable shape and can include guide holes or pulleys. The guides maintain the pulling wires 190 at 180 degrees from one another, which creates a plane in which the end section 140 can angulate along the formed single plane. The guide wires 190 are in direct contact with and move through or along guides 210 and extend to end section 140 where they are secured in any suitable manner, for instance, by welds or a mechanical fastener or throughhole.

The guides 210 facilitate movement of the pull wires laterally farther away from one another, as compared to their relative spacing within the elongated housing, as the second actuator is engaged to move and tension one of the pull wires. That is, an actuator is moved to pull one of the pulling wires towards the handpiece thereby shortening the pull wire as compared to the other pull wire. This movement of one pull wire leads to a moment arm, which causes end section 140 to actuate in an angular manner up to 90 degrees (e.g., the α- and β-angles described above). As such, the pull wires, by being tensioned one at a time, angulate the end section 140 and also the center wire 160 and tool 150 attached thereto. The end section 140 does not rotate about a mechanical hinge, however, the end section, in an angulated position, can be rotated by the rotation actuator described above secured to elongated housing and positioned at or near the handpiece of the instrument.

The pull wires 190, by being attached to one end of the end section 140, allow a bending moment to be positioned at or near the end of the instrument between end section 140 and guide section 200 as illustrated in FIGS. 6 and 7. The length of the end section 140 can be selected to ensure a small radius of angulation at the end of the instrument, for example, the end section 140 can be 5 centimeter (cm) or less, 3 cm or less, 1 cm or less, 0.8 cm or less, 0.6 cm or less, 0.5 cm or less, 0.4 cm or less, 0.3 cm or 0.2 cm or less. The flexible section between guide section 200 and end section 140 that bends up to 90 degrees along a single plane can have similar length as the end section, for instance, 5 cm or less, 3 cm or less, 1 cm or less, 0.8 cm or less, 0.6 cm or less, 0.5 cm or less, 0.4 cm or less, 0.3 cm or 0.2 cm or less. The overall tip portion 70 of the instrument can be of similar length and in the range of 8 cm or less, 6 cm or less, 5 cm or less, 4 cm or less, 3 cm or less or 2 cm or less. The elongated housing 60 connected to the guide section 200 of the tip portion 70, can be of any suitable length depending on the procedures conducted. For example, the elongated housing 60 can be 0.5 meter (m) to 5 meters, or 1 to 3 meters.

The tip portion 70 of the instrument, upon angulation should naturally tend to return to center position after single-plane actuation such that end section 140 is aligned with guide section 200. To assist the end section 140 to be in line with guide section 200 and promote travel along the single plane actuation as the pull wires are engaged, as shown in FIG. 8, a spring blade 170 can be positioned between guide section 200 and end section 140 of the tip portion 70. The spring blade 170 provides a structural connection between guide section 200 and end section 140 to ensure the tip portion 70 is rigid and not unintentionally flexing outside of a single plane unless an actuator is engaged. The spring blade 170 can be any suitable resilient member and can be comprised of or include one or more elastic metals, such as nitinol, plastic, elastomers, copolymers, or a combination thereof. The spring blade 170, in one example, can be secured to guide section 200 and end section 140, for instance by spot welds, fasteners or adhesive, and thus aid in forcing the sections 200, 140 to return to their natural position in line with one another. In another example, the ends 170a, 170b of spring blade 170 can extend into guide section 200 and end section 140 to both position the blade 170 between the sections and control the distance gap between section 140, 200. The spring blade 170 preferably includes a narrowed portion at or near its center 175. As secured to or fitted in end section 140, the material, shape and configuration of spring blade 170 allows end section 140 to bend, for instance around its narrowed center 175, and naturally straighten when no forces such as pull wires are acting on the end section. To accommodate bending along a single plane and provide a structural connection between sections 200 and 140, the spring blade 170 is made from a thin material, for example, the thickness of the spring blade can be in the range of 0.01 to 0.6 millimeter (mm), 0.03 to 0.5 mm, 0.04 to 0.4 mm, or 0.05 to 0.3 mm.

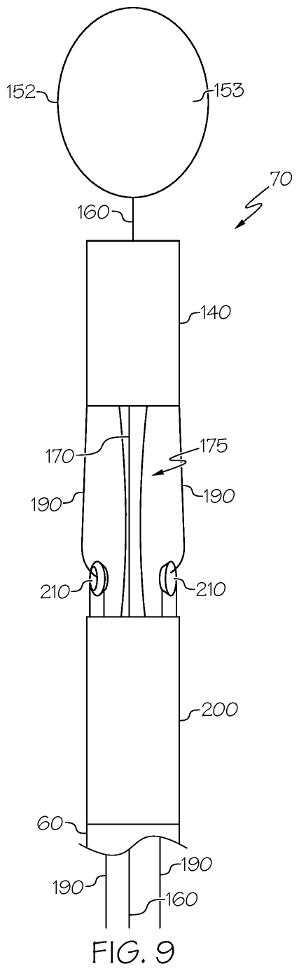

FIG. 9 shows another embodiment of a tool 152 that can be used with the tip portion 70 of the endoscopic instrument 10. The tip portion 70 includes an end section 140 having two ends, a through passage along its entire length, and an opening at each end for passing a center wire and tool through. Extending from the distal or first end of the end section 140 is tool 152, which can be extended and retracted by a first actuator moving a center wire 160 in a linear manner. The center wire 160 passes through elongated housing 60 and into end section 140, all of which can be covered with a protective sheath or covering. In the instance the center wire 160 is connected to an electrical source as described herein, such as for a hot procedure, an insulating sheath can be used to protect against any electrical current along the surface of the instrument, including the elongated housing and tip portion.

The tool 152 can include any desired configuration depending upon the procedure desired. As shown, the tool 152 can be a snare. A snare can grasp, dissect, and transect tissue during a procedure, for instance, gastrointestinal endoscopy procedures. The snare includes a retractable loop, for example, of metal wire, that can be extended from end section 140 to form a loop with an opening 153. The loop opening 153, during a procedure, can be partly or completed closed by retracting the tool 152 back into end section 140 with the first actuator and thereby reducing the size of the loop as desired. One end of the tool 152 can be secured, for example by a weld or the like, to the end of moveable center wire 160 that can, in certain instances, pass through end section 140 for extending and retracting the tool. The snare can be any suitable shape, such as a circle, oval, hexagon shaped, diamond shape and the like.

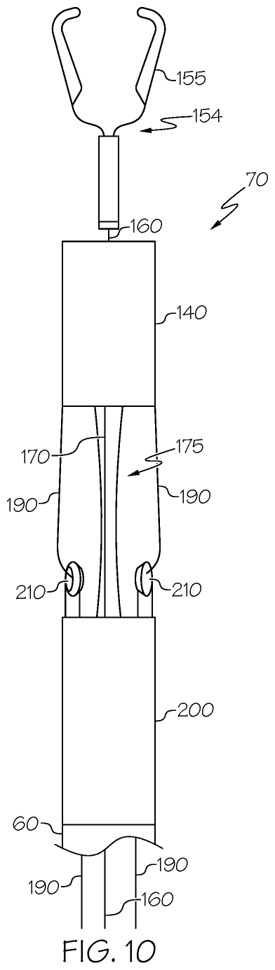

FIG. 10 shows another embodiment of a tool 154 that can be used with the tip portion 70 of the endoscopic instrument 10. As disclosed above, the tip portion 70 includes an end section 140 with a through passage along its entire length, and an opening at each end for passing a center wire 160 and tool 154 through. Extending from the distal or first end of the end section 140 is tool 154, which can be extended and retracted by a first actuator moving a center wire 160. The center wire 160 passes through elongated housing 60 and into end section 140, all of which can be covered with a protective sheath or covering all the way to the distal end of section 140 (not shown).

The tool 154 can include any desired configuration depending upon the procedure desired. As shown, the tool 154 can be a clip such as an endoscopic clip or endoclip. The clip is a metal mechanical device that can close or secure surfaces together, for example tissue in the gastrointestinal tract, without the need for surgery or suturing. The clip includes a pair of arms 155 that can open and close by actuating wire 160. FIG. 10 shows the arms 155 in an open position for positioning tool 154 at the desired location for securing two surfaces with one another. The first actuator can be engaged to close arms 154 and once the arms are closed and in place the first actuator can be further engaged to detach tool 154 from wire 160. The clip can be any suitable variation of shape and size, for example, the arms 155 can be different lengths and the open apart from one another at different spacings.

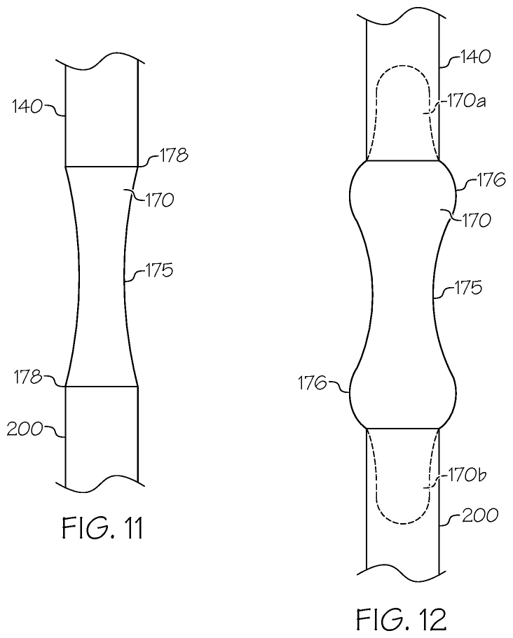

FIGS. 11 and 12 show examples of spring blades 170 that can be used with the instrument for single-plane articulation of the end section and tool. FIG. 11 shows a spring blade 170 embodiment having a first end secured to end section 140 and a second opposite end secured to guide section 200. Attachment areas 178 for securing spring blade 170 to sections 140, 200 are shown and can include welds, fasteners, epoxy or other adhesives. Spring blade 170 as shown can have a narrowed portion 175 at or near its center for accommodating easier bending upon actuation of end section 140. The narrowed portion 175 can have a width at its narrowest point that is less than the diameter of either end section 140 or guide section 200. The overall length of spring blade 170, measured from the connection points to sections 140, 200, can be in the range of 0.2 to 5 cm, 0.3 to 3 cm, 0.4 to 2 cm. The spring blade 170

FIG. 12 shows another example of spring blade 170. Spring blade 170 has a first end 170a extending into a portion of end section 140 and an opposite second end 170b extending into a portion of guide section 200. First and second ends 170a, 170b of spring blade 170 have a width that is less than the diameter of either end section 140 or guide section 200 to permit the ends to extend into the openings of both sections. Although not shown, center wire 160 and pull wires pass through guide section 200 and center wire 160 extends further and passes through end section 140. To prevent the main body portion of spring blade 170 from also extending into sections 140, 200, spring blade 170 has an expanded section, protrusion or barb 176 near each end that serves as stop or contact point with the sections. In operation, spring blade 170 bends and flexes while ends 170a and 170b remain positioned in sections 140, 200. Ends 170a and 170b also serve to provide force on sections 140 and 200 to naturally straighten each section when the pull wires are not being actuated. Similar to that shown in FIG. 11, spring blade 170 can also have a narrowed portion 175 at or near its center for accommodating easier bending upon actuation of end section 140.

Although embodiments described herein are made with reference to example embodiments, it should be appreciated by those skilled in the art that various modifications are well within the scope and spirit of this disclosure. Therefore, the scope of the example embodiments is not limited herein. The disclosure is intended to include all such modifications and alterations disclosed herein or ascertainable herefrom by persons of ordinary skill in the art without undue experimentation. It will be appreciated that an endoscopic instrument configured in accordance with the examples shown herein can be used for a wide variety of other procedures.

1. An endoscopic instrument comprising:
a center wire comprising a first end and a second end;
at least one pull wire having a first end and a second end;
a first actuator secured to the first end of the center wire, the first actuator configured to move the center wire in a linear manner;
a tool secured to the second end of the center wire;
a second actuator secured to the first end of the at least one pull wire, the second end of the at least one pull wire secured to an end section of the instrument; and
the second actuator is configured to angularly articulate the tool in a single plane by moving the at least one pull wire.

2. The endoscopic instrument of claim 1, wherein the second actuator can move the tool up to 90 degrees in the single plane.

3. The endoscopic instrument of claim 1, wherein the second actuator is secured to a second pull wire having a first end and a second end.

4. The endoscopic instrument of claim 3, wherein the second actuator is configured to angularly articulate the tool ±90 degrees in the single plane by moving at least one of the pull wires.

5. The endoscopic instrument of claim 3, wherein the pull wires are in direct contact with guides for positioning the pull wires at 180 degrees from one another.

6. The endoscopic instrument of claim 1, wherein the center wire is an electrode.

7. The endoscopic instrument of claim 1, wherein the tool is selected from a group consisting of an electrocautery knife, a snare and a clip.

8. The endoscopic instrument of claim 1, wherein a portion of the center wire and a portion of the at least one pull wire are positioned in an elongated housing, the elongated housing covered by an insulating sheath.

9. The endoscopic instrument of claim 1, further comprising a rotation actuator configured to rotate an end section, the end section comprising an open end configured to pass the tool therethrough.

10. The endoscopic instrument of claim 1, wherein the center wire comprises a flexible section positioned near the second end of the at least one pull wire.

11. The endoscopic instrument of claim 1, further comprising an electrical coupler for coupling the center wire to an electrical source.

12. The endoscopic instrument of claim 1, wherein a spring blade is positioned between a guide section and the end section, the spring blade is articulated ±90 degrees in the single plane by moving the at least one pull wire.

13. An endoscopic instrument comprising:
an elongated housing having a first end and a second end;
a center wire having a first end and a second end, a portion of the center wire positioned in the elongated housing;
a first actuator secured to the first end of the center wire, the first actuator configured to move the center wire in a linear manner;
a tool secured to the second end of the center wire, the tool configured to pass in and out of a first end of an end section by use of the first actuator;
a pair of pull wires, each pull wire having a first end and a second end, a portion of the pair of pull wires positioned in the elongated housing;
a second actuator secured to the first end of each of the pair of pull wires, the second end of each of the pair of pull wires secured to a second end of the end section; and
the second actuator is configured to angularly articulate the tool ±90 degrees in a single plane by moving the pair of pull wires.

14. The endoscopic instrument of claim 13, wherein the elongated housing being a coiled metal housing.

15. The endoscopic instrument of claim 1, wherein the pair of pull wires are in direct contact with guides for positioning the pair of pull wires at 180 degrees to form the single plane in which the end section can angulate.

16. The endoscopic instrument of claim 15, wherein the guides are secured to a guide section, the guide section connected to the second end of the elongated housing.

17. The endoscopic instrument of claim 13, wherein a spring blade is positioned between a guide section and the end section, the spring blade is articulated ±90 degrees in the single plane by moving the pair of pull wires.

18. The endoscopic instrument of claim 17, wherein the spring blade has a first end and a second end, the first end of the spring blade being secured to the second end of the end section and the second end of the spring blade being secured to the first end of a guide section.

19. The endoscopic instrument of claim 17, wherein the spring blade has a first end and a second end, the first end of the spring blade extends into the second end of the end section and the second end of the spring blade extends into the first end of a guide section.

20. The endoscopic instrument of claim 17, wherein the spring blade is configured to return to its unarticulated shape.

21. The endoscopic instrument of claim 13, wherein the tool is selected from a group consisting of an electrocautery knife, a snare and a clip.
